(19) United States
(12) Patent Application Publication
(10) Pub. No.: US 2007/0260315 A1
(43) Pub. Date: Nov. 8, 2007

(54) Devices and methods for disc height restoration

(76) Inventors: Kevin T. Foley, Germantown, TN (US); Roy Lim, Germantown, TN (US)

Correspondence Address: COATS & BENNETT, PLLC, 1400 Crescent Green, Suite 300, Cary, NC, 27518, US

(21) Appl. No.: 11/416,892

(22) Filed: May 3, 2006

(51) Int. Cl. A61F 2/44; A61B 17/88

(52) U.S. Cl. 623/17.12; 606/90

(57) ABSTRACT

Devices and methods to restore the disc height between adjacent vertebral members. The methods use a variety of different spacers that are each positionable between a first orientation having a reduced height and a second orientation having an enlarged height. The spacer is initially placed within the disc space in the first orientation. The spacer is then expanded to the second enlarged orientation to restore the disc height. While the spacer is expanded or while it is expanding, the material is inserted into the disc space. In one embodiment, the material is initially in a first flowable form that fills the disc space. After the material is inserted, it becomes more viscous to support the vertebral members. At this time, the spacer is returned to the first orientation and removed from the disc space. The material remains within the disc space to permanently maintain the disc height.

### BACKGROUND

A large majority of the population will experience back pain at some point in their lives that results from a spinal condition. The pain may range from general discomfort to disabling pain that immobilizes the individual. The back pain may result from a trauma to the spine, be caused by the natural aging process, or may be the result of a degenerative disease or condition.

Procedures to remedy these problems may require correcting the spacing between vertebral members. One or more spacing devices are positioned between the vertebral members and adjusted to the proper size. The devices used for gaining the correct spacing may permanently remain within the patient, or may be removed and replaced by other spacing means. The devices have a variety of shapes and sizes depending upon the application.

Some of these procedures may be performed in a minimally invasive manner. Minimally invasive techniques are advantageous because they can be performed with the use of a local anesthesia, have a shorter recovery period, result in little to no blood loss, and greatly decrease the chances of significant complications. Minimally invasive techniques additionally are usually less expensive for the patient.

### SUMMARY

The present application is directed to methods and devices to increase the disc height between adjacent vertebral members. Device embodiments may include a spacer positionable between a first orientation having a reduced size and a second orientation have an enlarged size. In some embodiments, a sheath is positioned around the spacer to prevent a material inserted into the disc space from contacting the spacer. In other embodiments, there is no sheath positioned around the spacer.

One method comprises placing the spacer within the disc space. The spacer is expanded to the second orientation to increase the disc height. While the spacer is expanded, the material is inserted into the disc space. After the material is inserted, the spacer is returned to the first orientation and removed from the disc space. The material remains permanently between the vertebral members to maintain the disc height.

### BRIEF DESCRIPTION OF THE DRAWINGS

FIG. 1 is a perspective view of a spacer in a closed orientation with a sheath extending around the spacer according to one embodiment.

FIG. 2 is a perspective view of a spacer in an open orientation with a sheath extending around the spacer according to one embodiment.

FIG. 3 is a side view illustrating a spacer and a sheath in the closed orientation inserted between vertebral members according to one embodiment.

FIG. 4 is a side view illustrating the spacer and sheath in the open orientation inserted between vertebral members according to one embodiment.

FIG. 5 is a side view of a material being inserted between the vertebral members according to one embodiment.

FIG. 6 is a side view of the spacer in a closed orientation being removed from the material according to one embodiment.

FIG. 7 is a cross-sectional view cut along line 7-7 of FIG. 6 illustrating the material within the disc space according to one embodiment.

FIG. 8 is a side view of another embodiment of a spacer according to one embodiment.

### DETAILED DESCRIPTION

The present invention is directed to devices and methods to restore the disc height between adjacent vertebral members. The methods use a variety of different spacers that are each positionable between a first orientation having a reduced height and a second orientation having an enlarged height. The spacer is initially placed within the disc space in the first orientation. The spacer is then expanded to the second enlarged orientation to restore the disc height. While the spacer is expanded or while it is expanding, the material is inserted into the disc space. In one embodiment, the material is initially in a first flowable form that fills the disc space. After the material is inserted, it becomes more viscous to support the vertebral members. At this time, the spacer is returned to the first orientation and removed from the disc space. The material remains within the disc space to permanently maintain the disc height.

The spacer 20 includes opposing support surfaces that are positioned a distance apart to define the overall height. The spacer 20 is adjustable between a first orientation having a first height and a second orientation having a second larger height. The reduced height of the first orientation allows the spacer 20 to be inserted and removed from the patient in a minimally-invasive manner. The second larger height causes the spacer 20 to increase the disc space 92 between vertebral members 90, 91 and restore the disc height, or return the disc height towards the normal size. A height control mechanism for adjusting the spacer height may be positioned remotely from the spacer 20 and monitored during the procedure to position the vertebral members 90, 91 at the proper spacing.

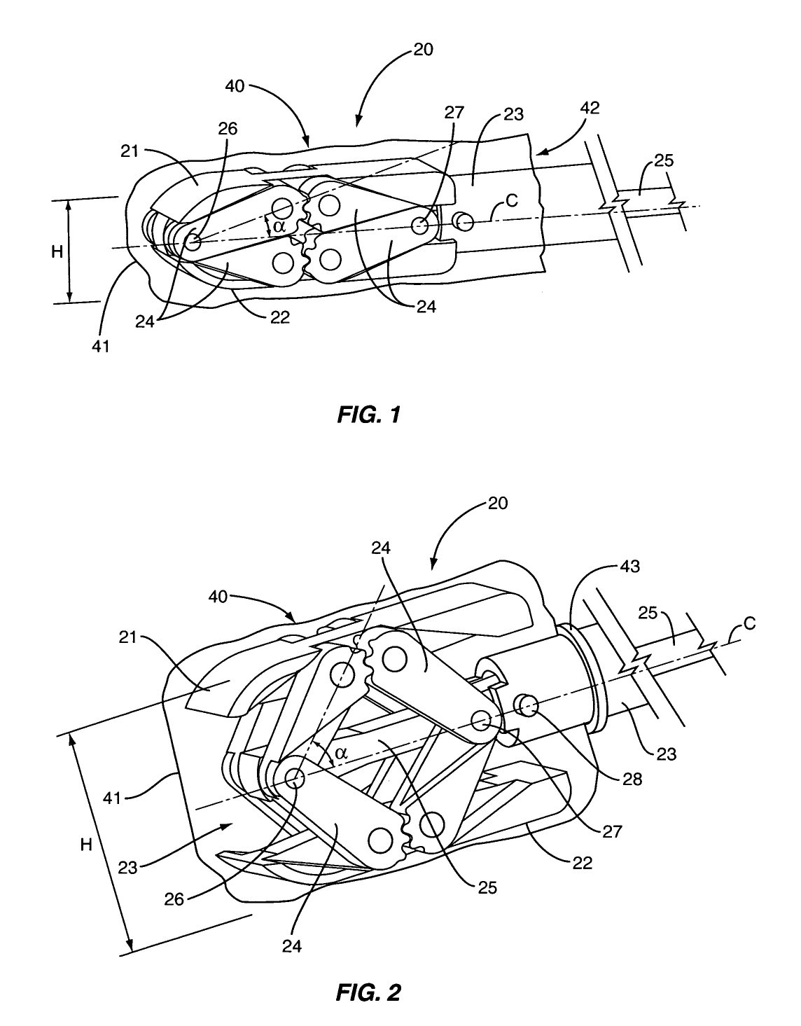

FIGS. 1 and 2 illustrate one embodiment of the spacer 20. In this embodiment, a sheath 40 is placed around the spacer 20, or a portion of the spacer 20. FIG. 1 illustrates the spacer 20 in the first orientation having a reduced height H, with FIG. 2 illustrating the second orientation with the enlarged second height H. The spacer 20 in this embodiment features an upper plate 21 and a lower plate 22 that define the height and extend between linkages 24. A pull arm 25 is positioned between the plates 21, 22 and moves to deploy the linkages 24 and control the height H. A first pin 26 attaches the distal linkages to the pull arm 25, and a second pin 27 attaches the proximal linkages to the pull arm 25. The pull arm 25 includes an elongated slot (not illustrated) through which the second pin 27 extends and connects the proximal linkages. In the closed orientation, the pull arm 25 is in a distal position with the first pin 26 and the second pin 27 spaced a first distance apart. During deployment, the pull arm 25 is moved proximally and the first pin 26 and inner ends of the distal linkages are likewise moved proximally. The second pin 27 is stationary because the pin 27 slides within the elongated slot. The proximal movement of the pull arm 25 reduces the distance between the pins 26, 27 causing the linkages 24 to unfold. The unfolding action moves the plates 21, 22 outward from the centerline C and increases the height H. The amount of proximal movement of the pull arm 25 controls the height H.

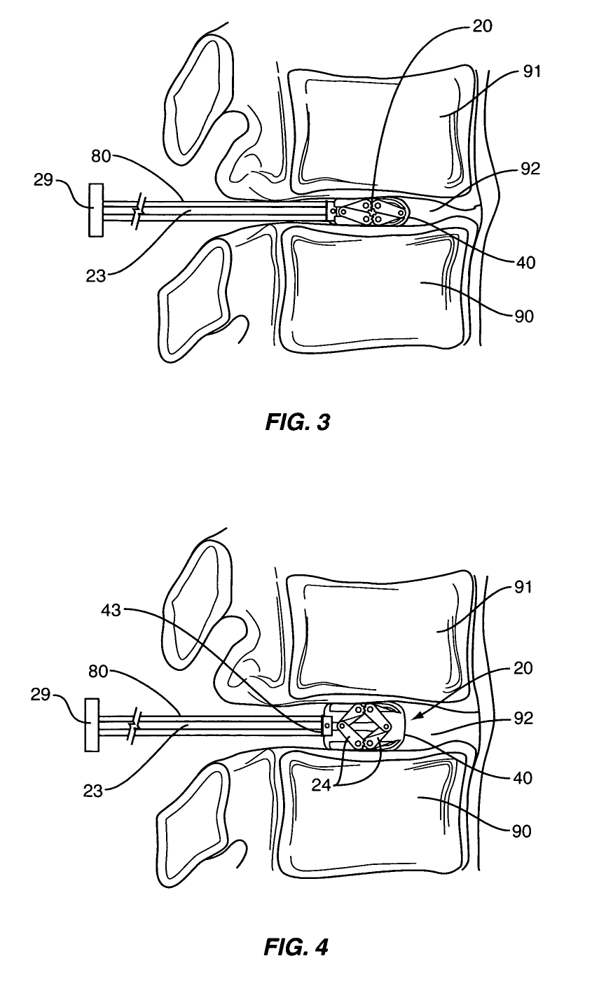

A delivery device 23 is connected to the spacer 20. The delivery device 23 has an elongated shape with the distal end attached to the spacer 20, and a proximal end spaced a distance away. The length of the delivery device 23 allows for the proximal end to be positioned outside of the patient when the spacer 20 is between the vertebral members 90, 91. A deploying mechanism 29 (FIGS. 3 and 4) mounted on the delivery device 23 causes movement of the pull arm 25 and thus is used to control the spacer height H. In one embodiment, deploying mechanism 29 is a knob operatively connected to the pull arm 25. Rotation of the knob moves the pull arm 25 relative to the delivery device 23 to control the height H.

Spacer 20 may be removably connected to the delivery device 23. In one embodiment, a connection member 28 connects the spacer 20 to the delivery device 23. In another embodiment, a distal end of the delivery device 23 includes threads that connect to corresponding threads on a proximal end of the spacer 20. Relative rotation of the device 23 and spacer 20 provides for attachment and detachment. In either embodiment, spacer 20 may remain connected to the delivery device 23 during the procedure, or may be removed after the spacer 20 is deployed between the vertebral members 90, 91. The delivery device 23 may then be reconnected to the spacer 20 for removal from the patient.

One embodiment of the spacer is disclosed in U.S. patent application Ser. No. 10/178,960 entitled “Minimally Invasive Expanding Spacer and Method” filed on Jun. 25, 2002, herein incorporated by reference in its entirety. Another embodiment is disclosed in U.S. patent application Ser. No. 10/817,024 that is a continuation-in-part of the '960 application, and is also incorporated by reference in its entirety.

In one embodiment, the sheath 40 extends around the spacer 20 and prevents the material 30 from directly contacting the spacer 20. FIGS. 1 and 2 illustrate an embodiment with the sheath 40 extending around the spacer 20. Sheath 40 includes a closed end 41 with an opening 42 positioned on an opposite side. A seal 43 closes the opening 42 and prevents entry of the material 30 into the interior of the sheath 40. The seal 43 may be integral with the sheath 40, or may be a separate member.

In another embodiment, sheath 40 extends around a limited portion of the spacer 20. In one example, sheath 40 extends around the distal end of the spacer 20. In one embodiment, sheath 40 extends around moving sections of the spacer 20 that allow for returning to the reduced sized. In one specific embodiment, sheath 20 extends around the linkages 24 and pins 26, 27. In another embodiment, sheath 40 extends along a portion of the entirety of the delivery device 23.

Sheath 40 may be constructed of a variety of materials. In one embodiment, sheath 20 is constructed of an elastic material that stretches as the spacer 20 moves from the first orientation to the second orientation. In another embodiment, the sheath 40 is constructed of a non-elastic material that has a fixed size that conforms to the dimension of the spacer 20 in the second orientation. In another embodiment, sheath 20 is constructed of a deformable material. Examples of sheath materials include polycarbonate urethane, polyurethane, silicon, and woven polyethylene.

FIG. 3 illustrates one embodiment of the spacer 20 in a reduced first orientation positioned within the disc space 92 between the vertebral members 90, 91. Prior to insertion, the diseased or damaged disc is removed, either wholly or in part, from between the vertebral members 90, 91. In one embodiment, the nucleus of the disc is removed and the annulus fibrosis remains within space 92. The proximal end of the delivery device 23 and deploying mechanism 29 are positioned outside of the patient to be accessed by the physician performing the procedure. In this embodiment, the sheath 40 extends around the spacer 20 and the opening 42 is sealed shut prior to insertion into the space 92.

The insertion of the spacer 20 into the disc space 92 may be facilitated by a cannula 80. The cannula 80 is inserted within a small incision made in the patient that extends to the disc space 92. In one embodiment, the cannula 80 is a METRx tube, available from Medtronic Sofamor Danek of Memphis, Tenn.

FIG. 4 illustrates the spacer 20 in the expanded second orientation. The linkages 24 have unfolded and the upper and lower plates 21, 22 contact the vertebral members 90, 91 and restore the disc space 92 to the proper size. The sheath 40 remains around the spacer 20.

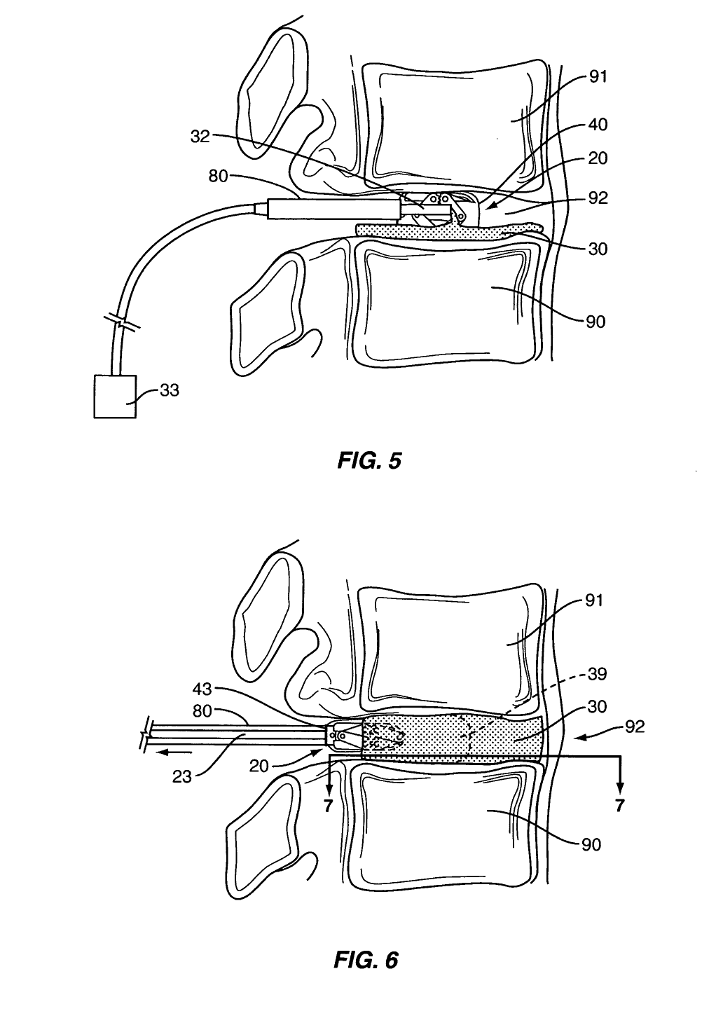

FIG. 5 illustrates an input mechanism 32 that introduces the material into the disc space 92. In one embodiment, the input mechanism 32 is sized to fit within the cannula 80. The input mechanism 32 may include a pump 33 to force the material 30 into the disc space 92. A pressure indicator (not illustrated) may also be associated with the input mechanism 32 to monitor the amount of pressure used for inputting the material 30. An indicator (not illustrated) may further be associated with the input mechanism 32 to indicate the amount of material placed within the space 92.

Material 30 is introduced in a first flowable form that spreads throughout the disc space 92. The amount of material 30 input into the disc space 92 may vary depending upon the application. In the embodiment illustrated in FIG. 5, the delivery device 23 has been removed from the spacer 20 to save space within the cannula 80 to allow the input mechanism 32 and/or material 30 to be input into the disc space 92. In one embodiment, the annulus fibrosis prevents the material 30 from spreading beyond the disc space 92. In another embodiment, a containment device is inserted around a section of entirety of the disc space to prevent material spread.

In one embodiment, the sheath 40 prevents the material 30 from contacting the spacer 20. Without the sheath 40, the material 30 may clog the spacer 20 and prevent the spacer from being returned to a reduced for removal from the disc space 92.

Material 30 has an initial viscosity to be moved from the input mechanism and into the disc space. Once within the disc space, the material 30 cures, meaning that it progresses from an initial flowable form during delivery to a more permanent form for final use in vivo. In one example, permanent form comprises a substantially rigid shape capable of maintaining a predetermined spacing between internal body components, such as bone. Material 30 may be a single component, or may include two or more different components that are mixed together prior to or during delivery. The material 30 may further be homogeneous with the same chemical and physical properties throughout, or heterogeneous. A variety of materials 30 may be used in the present invention and may include polyvinyl chlorides, polyethylenes, styrenic resins, polypropylene, thermoplastic polyesters, thermoplastic elastomers, calcium phosphate, calcium sulfate, polycarbonates, acrylonitrile-butadiene-styrene resins, acrylics, polyurethanes, nylons, styrene acrylonitriles, curable hydrogel, and cellulosics. Biomaterial may further include an opaque additive that will be visible on an X-ray. One type of additive includes barium sulfate.

The time necessary for the material 30 to harden may range from a few minutes to more than an hour. For a period of the hardening time, the spacer 20 remains in the open orientation to support the spacing of the vertebral members 90, 91. After a predetermined period of time, spacer 20 is moved towards the closed orientation and separates from contact with the vertebral members 90, 91.

FIG. 6 illustrates the removal of the spacer 20 from the disc space 92. The height of the spacer 20 is reduced causing the plates 21, 22 to move away from the vertebral members 90, 91. The spacer 20 is reduced to a height that fits within the cannula 80 and can be removed from the disc space 92. In one embodiment, prior to reducing the spacer height, the material 30 has hardened to a state that supports the vertebral members 90, 91 and maintains the disc height initially established by the spacer 20.

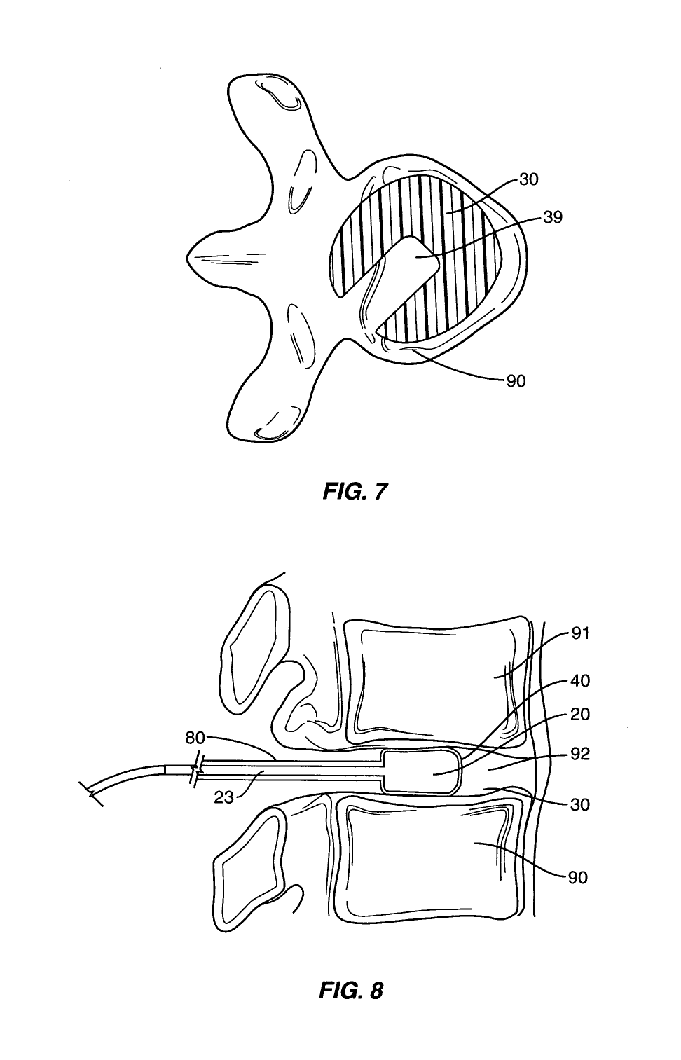

In one embodiment as illustrated in FIGS. 6 and 7, a void 39 is formed in the material 30 at the location of the spacer 20. The material 30 is substantially C-shaped when viewed in cross-section as illustrated in FIG. 7. One method further includes reinserting the input mechanism 32 through the cannula 80 and inputting additional material 30 to fill the void 39.

Various types of spacers 20 may be used in the present invention. The spacers 20 are each positionable between a first orientation with a first reduced height, and a second orientation with a second enlarged height. In some embodiments, spacer 20 may be able to be adjusted at different variations between the first and second orientations. In one embodiment, spacer 20 is remotely controlled to operate between the first and second orientations.

FIG. 8 illustrates another embodiment of a spacer 20 having an elastic balloon-like structure that can be inflated and deflated to control the height. A material is remotely inserted into and removed from the balloon-like structure to control the height.

In some embodiments, spacer 20 is directly inserted into the disc space 92 without a sheath 40. Spacer 20 is able to be selectively positioned between the first and second orientations. Further, the spacer 20 is able to be reduced to the smaller size after insertion of the material 30.

In some embodiments, spacer 20 is removed from the disc space 92 after insertion of the material. In one embodiment, this may occur well after the material 30 is able to independently support the vertebral members 90, 91. By way of example, a revision surgery is performed after an extended time period to remove the spacer 20. In another embodiment, spacer 20 is removed during the same procedure when the material 30 is introduced. This may be immediately upon the material 30 being able to independently support the vertebral members 90, 91, or at a later time. In one embodiment, spacer 20 remains permanently within the disc space 92 in the first, reduced orientation.

A variety of different input mechanisms 32 may be used for moving the material 30 into the disc space. One variety is a syringe-like device having a body for holding the material 30 and a plunger for forcing the material from the body and into the disc space. A scale may be printed on the body to visually determine the amount of expelled material that has been forced into the disc space.

In one embodiment, the sheath 40 has an elongated shape with the opening 42 positioned on the exterior of the patient when the spacer 20 is within the disc space 92 between the vertebral members 90, 91.

In one embodiment, the material 30 is started to be inserted into the disc space 92 prior to the spacer 20 being at the expanded, second orientation. The spacer 20 may be partially deployed towards the second orientation when the material 30 is initially inserted, or may still be at the first orientation. The spacer 20 is then moved towards the second orientation.

The term “distal” is generally defined as in the direction of the patient, or away from a user of a device. Conversely, “proximal” generally means away from the patient, or toward the user. Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting.

The present invention may be carried out in other specific ways than those herein set forth without departing from the scope and essential characteristics of the invention. These methods and devices may be used at a variety of locations along the spine including the cervical, thoracic, lumbar, and sacrococcygeal regions. Further, the approach to these areas of the spine may vary depending upon the application. The present embodiments are, therefore, to be considered in all respects as illustrative and not restrictive, and all changes coming within the meaning and equivalency range of the appended claims are intended to be embraced therein.

What is claimed is:

1. A method of spacing vertebral members comprising the steps of:
inserting a spacer within a disc space between the vertebral members;
expanding a height of the spacer to increase a distance between the vertebral members to form a disc height;
inserting a material in a first form into the disc space;
changing the material to a second form that is more viscous than the first form; and
after the material changes to the second form that maintains the disc height, reducing the height of the spacer and removing the spacer from the disc space.

2. The method of claim 1, wherein the step of changing the material to the second form that is more viscous than the first form comprises waiting a period of time for the material to harden.

3. The method of claim 1, further comprising introducing the spacer and the material into the disc space through a cannula.

4. The method of claim 1, further comprising prior to inserting the spacer into the disc space, inserting the spacer into a sheath and preventing contact between the material and the spacer.

5. The method of claim 4, further comprising increasing an interior volume of the sheath by expanding the height of the spacer.

6. The method of claim 1, further comprising forming the material into a substantially C-shaped member that is permanently positioned between the vertebral members.

7. The method of claim 1, further comprising inserting an additional amount of the material into the disc space after the spacer is removed.

8. The method of 1, wherein the steps of expanding and reducing the height of the spacer is performed remotely from the spacer.

9. The method of claim 1, wherein the step of reducing the height of the spacer and removing the spacer from the disc space comprises reducing the height of the spacer to be less than the disc height.

10. A method of spacing vertebral members comprising:
placing a spacer within a sheath;
inserting the spacer and the sheath into a disc space between the vertebral members;
expanding a height of the spacer and separating the vertebral members to an expanded height;
inserting a material between the vertebral members;
supporting the vertebral members at the expanded height with the material; and
reducing the height of the spacer to less than the expanded height and removing the spacer and the sheath from the disc space.

11. The method of claim 10, further comprising positioning a seal around the opening and closing the sheath around the spacer.

12. The method of claim 10, wherein the step of expanding the height of the spacer comprises expanding the sheath.

13. The method of claim 10, further comprising causing the material to acquire a second state that supports the vertebral members at the expanded height prior to reducing the height of the spacer to less than the expanded height.

14. The method of claim 13, wherein the step of reducing the height of the spacer occurs a predetermined time after the step of inserting the material between the vertebral members.

15. A method of spacing vertebral members comprising the steps of:
inserting a cannula to a disc space that is formed between the vertebral members, the cannula having a smaller height than the disc space;
inserting a spacer through the cannula and into the disc space;
expanding a height of the spacer and separating the vertebral members to increase the disc space;
inputting material in a first form through the cannula and into the disc space;
supporting the vertebral members with the material after the material has changed into a second form; and
thereafter, reducing the height of the spacer to fit within the cannula and removing the spacer from the disc space.

16. The method of claim 15, further comprising sealing the spacer within a sheath prior to inserting the spacer into the disc space.

17. The method of claim 15, wherein the step of removing the spacer from the disc space occurs after the material has changed to the second form that is more viscous than the first form.

18. The method of claim 15, wherein the steps of expanding the height of the spacer and reducing the height of the spacer are performed remotely from the disc space.

19. A method of spacing vertebral members comprising the steps of:
inserting a spacer within a disc space between the vertebral members;
expanding a height of the spacer to increase a distance between the vertebral members to form a disc height;
inserting a material in a first form into the disc space;
changing the material to a second form that is able to support the vertebral members at the disc height; and
after the material changes to the second form, reducing the height of the spacer and removing the spacer from the disc space.

20. The method of claim 19, wherein the step of changing the material to the second form comprises waiting a period of time for the material to harden.

21. The method of claim 19, further comprising introducing the spacer and the material into the disc space through a cannula.

22. The method of claim 19, further comprising prior to inserting the spacer into the disc space, inserting the spacer into a sheath and preventing contact between the material and the spacer.

23. The method of claim 19, further comprising forming the material into a substantially C-shaped member that is permanently positioned between the vertebral members.

24. The method of 19, wherein the steps of expanding and reducing the height of the spacer are performed remotely from the spacer.

25. A device to space vertebral members comprising:
a spacer positionable between a first orientation having a first height, and a second orientation having a second height greater than the first height;
a sheath that extends around the spacer and forms an interior environment within the sheath that is isolated from an exterior environment; and
an input mechanism to move a flowable material within the exterior environment between the vertebral members.

26. The device of claim 25, further comprising an elongated delivery device having a distal end that is attached to the spacer, and a proximal end spaced from the distal end, the distal end further comprising a removal means for removing the delivery device from the spacer.

27. The device of claim 25, wherein the sheath is constructed of an elastic material with the interior environment having a first volume when the spacer is in the first orientation, and a larger second volume when the spacer is in the second orientation.

28. The device of claim 25, wherein the sheath is of a fixed size and a volume of the interior environment is substantially constant when the spacer is in the first orientation and the second orientation.
